(19) United States
(12) Patent Application Publication
(10) Pub. No.: US 2018/0317484 A1
(43) Pub. Date: Nov. 8, 2018

(54) COMPOSITION FOR INHIBITING SYNTHESIS OF MYCOTOXIN WITHOUT AFFECTING THE GROWTH OF THE FUNGI

(71) Applicants: VIT University, Vellore (IN); BogaR Laboratories, Peddapuram Mandal (IN)

(72) Inventors: Alka Mehta, Vellore (IN); RameshBabu Boga, Peddapuram Mandal (IN); Dhanamjayulu P, Vellore (IN)

(21) Appl. No.: 15/958,092

(22) Filed: Apr. 20, 2018

(30) Foreign Application Priority Data
May 7, 2017 (IN) 201741016053

(51) Int. Cl. A01N 43/52; C07D 405/04

(52) U.S. Cl. CPC A01N 43/52; C07D 405/04

(57) ABSTRACT

A composition for inhibiting the growth of mycotoxins without affecting the growth of fungi is provided. The mycotoxin is an Aflatoxin and the fungus is an Aspergillus. The anti-aflatoxin composition comprises benzimidazole derivatives of Formula,

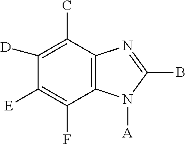

where the A is hydrogen or C1-C6 alkyl or C6-C14 aryl; B is C1-C10 alkyl or C6-C14 aryl or oxygen, sulfur, nitrogen containing heteroaryl; C and D are hydrogen or C1-C6 alkyl or C1-C6 alkoxyl or C6-C14 aryl; E is hydrogen or nitro or cyano or carboxyl or acetamidoxime or amidoxime or C1-C6 alkyl or C1-C6 alkoxyl or C6-C14 aryl or oxygen, sulfur, and nitrogen containing heteroaryl; F is hydrogen or C1-C6 alkyl or C1-C6 alkoxyl or C6-C14 aryl or oxygen, sulfur, and nitrogen containing heteroaryl, and mixture thereof with corresponding salts.

### CROSS-REFERENCE TO RELATED APPLICATIONS

This application claims priority to Indian patent application no. 201741016053 filed on May 7, 2017, the complete disclosure of which, in its entirely, is herein incorporated by reference.

### BACKGROUND

### Technical Field

The embodiments herein generally relate to a composition and method for inhibiting the synthesis of mycotoxin(s) without affecting the growth of the fungi, and more particularly aflatoxin producing fungi.

### Description of the Related Art

Mycotoxins occur as natural and unavoidable contaminants on a variety of food commodities. The Food and Agricultural Organization has reported that about 25% of the world's food crops are contaminated with mycotoxins. The ubiquitous nature of fungi makes food crops vulnerable to fungal contamination during pre-harvest and post-harvest conditions. From field to plate, food undergoes several stages of processing such as harvest, storage, transportation etc., and at every stage there is a risk of contamination by toxigenic fungi and their mycotoxins. Aflatoxins are a group of structurally related metabolites produced by Aspergillus flavus, Aspergillus parasiticus and Aspergillus nominus. These types of fungi can grow and produce aflatoxins under favorable temperature and moisture conditions in various food materials such as food grains, groundnut, tree nuts, dried fruits, spices, and herbs. These fungi are toxic to human and animals and causing severe economic losses due to rejection of contaminated food and feed. Further, these fungi, Aspergillus flavus, Aspergillus parasiticus and Aspergillus nominus are used in fermentation food processing techniques where the inhibition of fungi is not possible and at the same time the effect of toxins produced by these fungi creates a major health issue. Hence, the inhibition of the aflatoxin production without killing the fungi is to be targeted for research to overcome the aforementioned problems.

Chemical control, using currently available fungicides, is one of the widely used ways for preventing mycotoxigenic fungal growth and reducing mycotoxin contamination. Guerra et al., in their work titled “Evaluation of antifungal activity and mode of action of new coumarin derivative, 7-hydroxy-6-nitro-2H-I-benzopyran-2-one, against Aspergillus spp.” published in Evidence-Based Complementary and Alternative Medicine, (Volume 2015, Article ID 925096, 8 pages), and used coumarin derivative, 7-hydroxy-6-nitro-2H-I-benzopyran-2-one as an antifungal agent against Aspergillus species. Ono et al., presented Aflastatin A, a novel inhibitor of the aflatoxin production by aflatoxigenic fungi, has been isolated from the mycelial cake of Streptomyces in their paper titled “Aflastatin A, a novel inhibitor of aflatoxin production by aflatoxigenic fungi” published in The Journal of antibiotics 1997, 50(2): 111-118. On the other hand, benzodioxole and eugenol suppressed AFB1 production without AFG formation, while methyleugenol showed inhibition of AFB1 production with slight production of AFG1. Moon et al, in their work titled “Antifungal and anti-aflatoxigenic methylenedioxy-Containing compounds and piperine-like synthetic compounds” showed that Piperonal and 1,3-benzodioxole had inhibitory effects against A. flavus mycelial growth and aflatoxin B1 production which is published in Toxins 2016, 8(8):240.

Bhatnagar et al., 1988 in their work titled “The inhibitory effect of neem (Azadirachta indica) leaf extracts on aflatoxin synthesis in Aspergillus parasiticus” published in Journal of the American Oil Chemists' Society 1988, 65(7): 1166-1168, and Allameh et al., in their work titled “Effects of neem leaf extract on production of aflatoxins and activities of fatty acid synthetase, isocitrate dehydrogenase and glutathione S-transferase in Aspergillus parasiticus” published in Mycopathologia 2002, 154(2):79-84, and previously reported that biological components of a neem tree i.e., Azadirachta indica are well known for its interference in aflatoxin biosynthesis with very little action on the fungal mycelia. Most of the literature reported the antifungal activity and that leads to inhibition of mycotoxins. However, very few are efficient in controlling Aflatoxin effectively without affecting the growth of the fungi, A. flavus. Thus, there is a need for a compound/composition that can effectively inhibit aflatoxin production without impairing the fungal growth.

### SUMMARY

In view of a foregoing, the present disclosure provides a composition for inhibiting synthesis of mycotoxins without affecting the growth of the fungi including a benzimidazole compound of Formula:

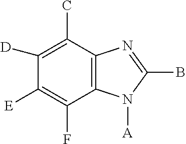

where the A is hydrogen or C1-C6 alkyl or C6-C14 aryl; B is C1-C10 alkyl or C6-C14 aryl or oxygen, sulfur, nitrogen containing heteroaryl; C and D are hydrogen or C1-C6 alkyl or C1-C6 alkoxyl or C6-C14 aryl; E is hydrogen or nitro or cyano or carboxyl or acetamidoxime or amidoxime or C1-C6 alkyl or C1-C6 alkoxyl or C6-C14 aryl or oxygen, sulfur, and nitrogen containing heteroaryl; F is hydrogen or C1-C6 alkyl or C1-C6 alkoxyl or C6-C14 aryl or oxygen, sulfur, and nitrogen containing heteroaryl, and mixture thereof with corresponding salts.

The present disclosure also provides a method for inhibiting the growth of mycotoxins without affecting the growth of fungi is provided. The mycotoxin is Aflatoxin and a fungus is Aspergillus. The method includes a chemical or pharmaceutical composition of benzimidazole derivatives as active compounds, having the following formula:

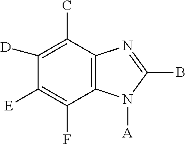

The present disclosure also provides an anti-aflatoxin composition including one or more active compounds selected from a group including:
(a) 2-(2-Furyl)-6-nitrobenzimidazole,
(b) 2-(2-Furyl)benzimidazole,
(c) 2-(2-Furyl)benzimidazole-6-carbonitrile,
(d) 2-(2-Furyl)benzimidazole-6-carboxamidoxime,
(e) 2-(2-Furyl)benzimidazole-6-carboxylic acid, and
(f) Combinations thereof.

The present disclosure further provides a method for inhibiting the synthesis of mycotoxin without affecting the growth of the fungi by applying a composition comprising an active compound of benzimidazole, wherein the process for the preparation of benzimidazoles comprising the steps of:

i) Corresponding derivatives of 1,2-phenylenediamine reacts with corresponding derivative of aldehyde, acid, or acid chloride with or without solvent (nitrobenzene) to obtain the corresponding benzimidazole derivative of formula

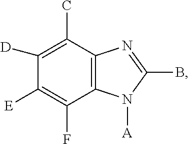

and
ii) Purification of obtained benzimidazole derivative from above step (i) by crystallization, or column chromatography to obtain the active compound Formula of acceptable derivative of anti-aflatoxin for formulation.

Embodiments of the present disclosure substantially eliminate or at least partially address the aforementioned problems in the prior art, and inhibits the mycotoxin synthesis without affecting much the growth of the fungi, using an anti-aflatoxin compounds, and thus eliminating the problem of toxin contamination during usage of Aspergillus like fungi in fermentation based food production processes.

Additional aspects, advantages, features and objects of the present disclosure are made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIGS. 1A-1C are schematic and graphical representations of thin-layer chromatogram, Dose-response overlay IR Spectrum and proton nuclear magnetic resonance (¹H-NMR) Spectrum of a benzimidazole derivative, 2-(2-Furyl)benzimidazole (FBD) respectively, in accordance with an embodiment of the present disclosure;

FIGS. 2A-2C are schematic and graphical representation of thin-layer chromatogram, Dose-response overlay IR spectrum and ¹H-NMR spectrum of 2-(2-Furyl)-6-nitrobenzimidazole (6-NFBD) respectively, in accordance with an embodiment of the present disclosure;

FIGS. 3A-3C are schematic and graphical representation of thin-layer chromatogram, Dose-response overlay IR spectrum and ¹H-NMR spectrum of 2-(2-Furyl)benzimidazole-6-carbonitrile (6-CFBD) respectively, in accordance with an embodiment of the present disclosure;

FIGS. 4A-4C are schematic and graphical representation of thin-layer chromatogram, Dose-response overlay IR spectrum and ¹H-NMR spectrum of 2-(2-Furyl)benzimidazole-6-carboxamidoxime (6-AFBD) respectively, in accordance with an embodiment of the present disclosure; and

FIGS. 5A-5B are schematic and graphical representation of thin-layer chromatogram, Dose-response overlay IR spectrum and 1H-NMR spectrum of 2-(2-Furyl)benzimidazole-6-carboxylic acid (6-CAFBD) respectively, in accordance with an embodiment of the present disclosure.

FIG. 6 is a tabular view illustrating an inhibition activity on growth of Aspergillus flavus in yeast extract sucrose (YES) medium in accordance with an embodiment of the present disclosure;

FIG. 7 is a graphical representation of a percentage of inhibition of Aspergillus flavus growth and aflatoxin production in yeast extract sucrose (YES) medium in accordance with an embodiment of the present disclosure;

FIG. 8 is a graphical representation of Fluorescence overlay chromatogram showing inhibition of an aflatoxin B1 (AFB1) in YES medium in accordance with an embodiment of the present disclosure;

FIG. 9 is a schematic representation of thin layer chromatogram showing an aflatoxin B1 (AFB1) spots in accordance with an embodiment of the present disclosure;

FIGS. 10A-10G is a microscopic view of a HeLa cell line cultured with test compound in accordance with an embodiment of the present disclosure;

FIG. 11 is a flowchart illustrating a method of preparation of an active compound in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

The present disclosure provides a composition for inhibiting synthesis of mycotoxins without affecting the growth of the fungi comprising a benzimidazole compound of Formula:

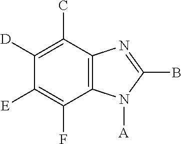

where the A is hydrogen or C1-C6 alkyl or C6-C14 aryl; B is C1-C10 alkyl or C6-C14 aryl or oxygen, sulfur, nitrogen containing heteroaryl; C and D are hydrogen or C1-C6 alkyl or C1-C6 alkoxyl or C6-C14 aryl; E is hydrogen or nitro or cyano or carboxyl or acetamidoxime or amidoxime or C1-C6 alkyl or C1-C6 alkoxyl or C6-C14 aryl or oxygen, sulfur, and nitrogen containing heteroaryl; F is hydrogen or C1-C6 alkyl or C1-C6 alkoxyl or C6-C14 aryl or oxygen, sulfur, and nitrogen containing heteroaryl, and mixture thereof with corresponding salts.

The present composition including benzimidazole may be used in various fermentation based food processing techniques like production of cheese, wine (sake), miso, natto etc. in which employs many Aspergillus and Penicillium species. In these processes, the fungal growth should not be inhibited but at the same time, the toxic compounds produced by these fungi during fermentation are to be inhibited. The present composition effectively inhibits the aflatoxin specifically, Aflatoxin B1 produced by Aspergillus flavus without disturbing the growth of the fungi.

### Definitions

The term alkyl broadly refers to a saturated linear monovalent hydrocarbon moiety of one to twelve, preferably one to six, carbon atoms or a saturated branched monovalent hydrocarbon moiety of three to twelve, preferably three to six, carbon atoms. Exemplary alkyl group include, but are not limited to, methyl, ethyl, n-propyl, 2-propyl, tert-butyl, pentyl, and the like. Here, alkyl refers to and covers any and all groups which are known as normal alkyl, branched-chain alkyl and cycloalkyl.

Aryl groups are cyclic aromatic hydrocarbons that do not contain heteroatoms. The aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In example embodiments, the aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

The term “heteroaryl” includes any compound having an aromatic ring which comprises a hetero atom. Here, the term “heteroaryl” means a monovalent monocyclic or bicyclic aromatic moiety of 5 to 12 ring atoms containing one, two, or three ring heteroatoms selected from N, O, or S, the remaining ring atoms being C. When the heteroaryl is polycyclic, at least one of the rings is aromatic. The nitrogen atoms may be in the form of N-oxides. By way of example of monocyclic heteroaryls, mention may be made of thiazole, thiadiazole, thiophene, imidazole, triazole, tetrazole, pyridine, furan, oxazole, isoxazole, oxadiazole, pyrrole, pyrazole, pyrimidine, pyridazine and pyrazine. By way of example of polycyclic heteroaryls, mention may be made of indole, benzofuran, benzimidazole, benzothiophene, benzotriazole, benzothiazole, benzoxazole, quinoline, isoquinoline, indazole, quinazoline, phthalazine, quinoxaline, naphthyridine, 2,3-dihydro-1H-indole, 2,3-dihydrobenzofuran, tetrahydroquinoline, tetrahydro-isoquinoline or tetrahydroisoquinazoline.

The term “alkoxyl” embraces linear or branched oxy-containing radicals each having acyl portions of one to about ten carbon atoms, such as a methoxyl group.

An effective acceptable salt may be prepared for any compounds of the present disclosure having functionality capable of forming such salt, for example an acid functionality. A pharmaceutically acceptable salt is any salt which retains the activity of the compound of the present disclosure and does not impart any deleterious or untoward effect on the subject to which it is administered and in the context in which it is administered.

According to an embodiment, when B is 2-furyl and A, C, D, E, F are hydrogen (—H), the compound of Formula is 2-(2-Furyl)benzimidazole (FBD) as follows:

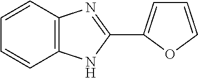 [SMILES: c1coc(-c2nc3ccccc3[nH]2)c1]

The molecular weight of FBD is 184.21. The melting point (MP) of FBD is 200° C. (degrees Celsius).

In an embodiment, when B is 2-furyl, E is nitro (—NO2) and A, C, D, F are hydrogen (—H), the compound of Formula is 2-(2-Furyl)-6-nitrobenzimidazole (6-NFBD) as follows:

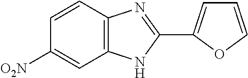 [SMILES: O=[N+]([O-])c1ccc2nc(-c3ccco3)[nH]c2c1]

The molecular weight of 6-NFBD is 229.21. The melting point (MP) of 6-NFBD is 195-200° C. (degrees Celsius).

In an embodiment, when B is 2-furyl, E is carbonitrile (—CN) and A, C, D, F are hydrogen (—H), the compound of Formula is 2-(2-Furyl) benzimidazole-6-carbonitrile (6-CFBD) as follows:

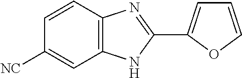 [SMILES: N#Cc1ccc2nc(-c3ccco3)[nH]c2c1]

The molecular weight of 6-CFBD is 209.22. The melting point (MP) of 6-CFBD is 215-220° C. (degrees Celsius).

In an embodiment, when B is 2-furyl, E is carboxyl (—COOH) and A, C, D, F are hydrogen (—H), the compound of Formula is 2-(2-Furyl) benzimidazole-6-carboxylic acid (6-CAFBD) as follows:

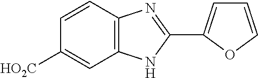 [SMILES: O=C(O)c1ccc2nc(-c3ccco3)[nH]c2c1]

The molecular weight of 6-CAFBD is 228.22. The melting point (MP) of 6-CAFBD is 200-204° C. (degrees Celsius).

In an embodiment, when B is 2-furyl, E is acetamidoxime (—C(NH2)=NOH) and A, C, D, F are hydrogen (—H), the compound of Formula is 2-(2-Furyl) benzimidazole-6-carboxamidoxime (6-AFBD) as follows:

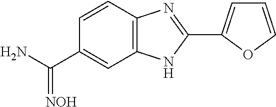

The molecular weight of 6-AFBD is 242.26. The melting point (MP) of 6-AFBD is 215-220° C. (degrees Celsius).

### Materials and Methods

The composition of the present disclosure includes a benzimidazole or its derivatives as active compounds to inhibit the synthesis of mycotoxins without affecting the growth of the fungi, Aspergillus flavus (A. flavus). Among the various active compounds, such as 2-(2-Furyl)benzimidazole (FBD), 2-(2-Furyl)-6-nitrobenzimidazole (6-NFBD), 2-(2-Furyl)benzimidazole-6-carbonitrile (6-CFBD), 2-(2-Furyl)benzimidazole-6-carboxamidoxime (6-AFBD), and 2-(2-Furyl)benzimidazole-6-carboxylic acid (6-CAFBD) may be used to study their effect against Aspergillus flavus and aflatoxin B1 (AFB1) production in a culture medium, yeast extract sucrose medium (YES). Each active compound with a concentration of 50 μg/ml is prepared in a 10 ml YES medium. Spores of the A. flavus may be harvested from a 9 day old culture on potato dextrose agar and counted using a hemocytometer. Fifty μl of a spore suspension (10⁵ spores/ml) is inoculated into each flask containing one active compound respectively. The flasks are incubated at 27±2° C. for a period of 7 days. At the end of the incubation, a dry mycelia weight (DMW) of the A. flavus and aflatoxin AFB1 production in the spent media (resulted) is determined. Then, the A. flavus is harvested by filtration through Whatman no. 1 filter paper and a broth thus obtained is used for extraction of AFB1. The wet weight of the A. flavus is recorded, and then dried at 60° C. in hot air oven until it reaches a constant weight. The spent media is extracted with equal volume of chloroform and the extraction is done thrice using separating funnel by wrist action. The extracts thus obtained from each spent medium is pooled and analyzed by thin layer chromatography (TLC), UV-Vis spectrophotometer and florescence spectroscopy for AFB1 production. Ten microliter of the chloroform extract from a test and a control along with a AFB1 standard is spotted on TLC (silica gel on aluminum foil with F 254 from Fluka, Munich, Germany) and is placed in a tank saturated with the solvent chloroform: acetone in the volume proportion of 85:15. The chromatograms are visualized under UV light. For the spectrophotometric analysis, extracts are suitably diluted and absorbance spectrum is obtained by UV-vis spectrophotometer (Shimadzu UV 1800, Kyoto, Japan). The AFB1 concentration is estimated by the absorbance at 365 nm.

To study the safety of the active compounds, each of them are subjected to cytotoxicity assay on HeLa cell line. HeLa cell line is obtained from NCCS Pune, India. MTT (3-(4, -dimethylthiazolyl-2)-2, 5-diphenyltetrazolium bromide) colorimetric method given by Mossman (1983) and modified by Trivedi et al. (1990) is used for the cytotoxicity assay. In brief, the active compounds are dissolved in dimethyl sulfoxide (DMSO) and further diluted with Dulbecco's modified Eagle's medium (DMEM, HiMedia, India). Then, 100 μl of this sample is added to the 96-well microplate. To each well 100 μl of HeLa cell suspension of 1×10⁵ cells/mL is added. Plates are incubated at 37° C. under 5% carbon dioxide and 95% atmospheric air. The DMSO concentration is kept below 0.1% in the samples. Observations are taken after 48 hours of incubation. The cell growth is measured in terms of color development due to formation of formazan from reduction of MTT by live cells. The absorbance is measured with microplate reader (Bio-Rad 680, USA) at 540 nm with reference wavelength of 600 nm. The percentage cytotoxicity is calculated as follows:

Cytotoxicity %=(1−absorbance of treated wells/absorbance of untreated wells)×100.

The present invention provides a method for inhibiting the synthesis of mycotoxins without affecting the growth of fungi. In one embodiment, the mycotoxin is Aflatoxin and a fungus is Aspergillus. The method involves a chemical or pharmaceutical composition of benzimidazole derivatives as active compounds, having the following formula.

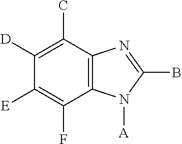

In one embodiment, the benzimidazole derivatives are obtained when the each position from A to E represent a functional group.

According an embodiment, in the active compound of Formula, B is 2-furyl and A, C, D, F are hydrogen (—H) and E is selected from a group of comprising hydrogen (—H), nitro group (—NO2), carbonitrile (—CN) group, carboxamidoxime (—C(NH2)=NOH) group and carboxylic acid (—COOH) group. In one embodiment, the benzimidazole derivatives or the active compounds include the combinations of derivatives that are having different functional groups.

The present disclosure also provides an anti-aflatoxin composition comprising one or more active compounds selected from a group comprising (a) 2-(2-Furyl)-6-nitrobenzimidazole, (b) 2-(2-Furyl)benzimidazole, (c) 2-(2-Furyl)benzimidazole-6-carbonitrile, (d) 2-(2-Furyl)benzimidazole-6-carboxamidoxime, (e) 2-(2-Furyl)benzimidazole-6-carboxylic acid or (f) combinations thereof.

In one embodiment, the anti-aflatoxin composition is (a) 2-(2-Furyl)-6-nitrobenzimidazole. In another embodiment, the anti-aflatoxin composition is (a) 2-(2-Furyl)-6-nitrobenzimidazole and (b) 2-(2-Furyl)benzimidazole. In another embodiment, the anti-aflatoxin composition is (a), (b) and (c). In yet another embodiment, the anti-aflatoxin composition is (a), (b), (c) and (d). In yet another embodiment, the anti-aflatoxin composition is (a), (b), (c), (d) and (e). In yet another embodiment, the anti-aflatoxin composition is a combination of any possible mixture including two or more active compounds of (a), (b), (c), (d) and (e).

According to an embodiment, the 2-(2-Furyl)-6-nitrobenzimidazole ranges from 0.01%-60%. According to another embodiment, the 2-(2-Furyl) benzimidazole ranges from 0.01%-60%. According to yet another embodiment, the 2-(2-Furyl) benzimidazole-6-carbonitrile ranges from 0.01%-60%. According to yet another embodiment, the 2-(2-Furyl) benzimidazole-6-carboxamidoxime ranges from 0.01%-60%. According to yet another embodiment, the 2-(2-Furyl) benzimidazole-6-carboxylic acid ranges from 0.01%-60%. In yet another embodiment, the anti-aflatoxin composition includes a therapeutically effective amount of at least one of 2-(2-Furyl)-6-nitrobenzimidazole, 2-(2-Furyl)benzimidazole, 2-(2-Furyl)benzimidazole-6-carbonitrile, 2-(2-Furyl)benzimidazole-6-carboxamidoxime, 2-(2-Furyl)benzimidazole-6-carboxylic acid, or combinations thereof.

According another embodiment, the anti-aflatoxin composition further comprises at least one of diluents, additives or any other carrier which is compatible with the at least one of the active compound.

As these active compounds are effective at very low concentrations (μg/mL) level, they have good utility for the prevention of aflatoxin and other mycotoxin synthesis during storage and transportation of food/agro products. Further these compounds showed no cytotoxicity on animal cell line when tested by 3-(4,5-dimethylthiazol-2-yl)-2,5-diphenyltetrazolium bromide (MTT) assay.

In one embodiment, the anti-aflatoxin composition is formulated as liquids, gels, sprays, powders, crystals, pellets, granules, emulsions or seed forms or its derivative thereof. In one embodiment, the anti-aflatoxin composition is impregnated as sachet, packaging material, patch, or air-freshener. The formulation of the anti-aflatoxin composition is prepared and delivered to the end process in such a way that it is easy and compatible to use in the Agro-based industry, fermented products and food processing industries.

The present disclosure further provides a method for inhibiting the synthesis of mycotoxin without affecting the growth of the fungi by applying a composition including an active compound comprising a benzimidazole. The process for the preparation of benzimidazoles including the steps of:

i) Corresponding derivatives of 1,2-phenylenediamine reacts with corresponding derivative of aldehyde, acid, or acid chloride with or without solvent (nitrobenzene) to obtain the corresponding benzimidazole derivative of formula

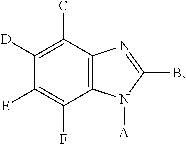

and
ii) Purification of obtained benzimidazole derivative from above step (i) by crystallization, or column chromatography to obtain the active compound Formula of acceptable derivative of anti-aflatoxin for formulation.

In one embodiment, the process for preparing the benzimidazole derivative includes the steps of: the derivatives of 1,2-phenylenediamine reacts with corresponding derivative of aldehyde, acid, or acid chloride with or without solvent (nitrobenzene) to obtain the corresponding benzimidazole derivative is as follows:

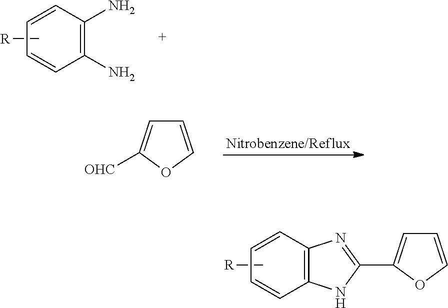

Where R represents the functional group of various derivatives of 1,2-phenylenediamine. In one embodiment, derivatives of 1,2-phenylenediamine is selected from a group, but not limited to, including 3,4-Diaminonitrobenzene, 3,4-Diaminobenzonitrile, 3,4-Diaminobenzoic acid or 1,4-dioxane.

In one embodiment, the benzimidazole derivative is selected from a group including 2-(2-Furyl)-6-nitrobenzimidazole, 2-(2-Furyl)benzimidazole, 2-(2-Furyl)benzimidazole-6-carbonitrile, 2-(2-Furyl)benzimidazole-6-carboxamidoxime, 2-(2-Furyl)benzimidazole-6-carboxylic acid and combinations thereof.

The following examples describe the preparation of the active compounds in accordance with the present disclosure. These examples are not limiting and merely illustrate the present disclosure.

### Example 1

A process for preparing the benzimidazole derivative, 2-(2-Furyl) benzimidazole (FBD) by reacting 1,2-phenylenediamine (0.1 mole) with Furfural (0.12 mole) in nitrobenzene solvent and the reaction mixture refluxed with stirring for 10 to 12 hours. Then, the reaction mixture was cooled and poured into ice cold to obtain solid of crude benzimidazole derivative, which is crystallized in Ethyl acetate and chloroform (1:1) to obtain a crystalline 2-(2-Furyl)-6-benzimidazole (FBD). The yield of FBD is 80%. The proton nuclear magnetic resonance (¹H-NMR) results including chemical shift, 8 (given in parts per million) that indicates the type of protons present in the active compound, FBD is given as follows: δ is 6.6-8.0 indicating the presence of aromatic protons, 7H, and δ is 13.0 indicating the presence of imidazole amine-NH, 1H. The solvent used is dimethyl sulfoxide (DMSO-d6). ¹H-NMR spectra may be recorded on a Varian-300 NMR or a Varian NMRS 500 spectrometer. Deuterated dimethyl sulfoxide-d6 may be used as the solvent. The resonances of non-deuterated dimethyl sulfoxide peaks at δ2.5 ppm and δ3.51 ppm were used as internal reference for ¹H-NMR spectra.

### Example 2

An equimolar amount of 3,4-diaminonitrobenzene is substituted for the 1,2-pheylenediamine in the preparation of Example 1 and the obtain product of 2-(2-Furyl)-6-nitrobenzimidazole (6-NFBD). The yield of 6-NFBD is approximately 82%. The proton nuclear magnetic resonance (¹H-NMR) results including chemical shift, δ (given in parts per million) that indicates the type of protons present in the active compound, 6-NFBD is given as follows: δ is 6.8-8.5 indicating the presence of aromatic protons, 6H, and δ is 13.6 indicating the presence of broad singlet for imidazole-NH, 1H. The solvent used is dimethyl sulfoxide (DMSO-d6).

### Example 3

An equimolar amount of 3,4-diaminobenzonitrile is substituted for the 1,2-pheylenediamine in the preparation of Example 1 and the obtain product of 2-(2-Furyl)benzimidazole-6-carbonitrile (6-CFBD). The yield of 6-CFBD is approximately 90%. The proton nuclear magnetic resonance (¹H-NMR) results including chemical shift, δ (given in parts per million) that indicates the type of protons present in the active compound, 6-CFBD is given as follows: δ is 6.6-8.2 indicating the presence of aromatic protons, 6H, and δ is 13.6 indicating the presence of broad singlet for imidazole-NH, 1H. The solvent used is dimethyl sulfoxide (DMSO-d6).

### Example 4

An equimolar amount of 3,4-diaminobenzoic acid is substituted for the 1,2-pheylenediamine in the preparation of Example 1 and the obtain product of 2-(2-Furyl)benzimidazole-6-carboxylic acid (6-CAFBD). The yield of 6-CAFBD is approximately 80%. The Infrared spectroscopy (IR) using Potassium bromide (KBr) pellets indicates the spectrum region of 1680 cm⁻¹ indicating the presence of carbonyl group in the active compound, 6-CAFBD.

### Example 5

A process for preparing the benzimidazole derivative, 2-(2-Furyl)benzimidazole-6-carboxamidoxime (6-AFBD) by reacting the synthesized active compound, 6-CFBD in Example 3, with hydroxylamine solution (20-30% in water) in 1,4-dioxane solvent and stirred the reaction mixture for 12 hours. Evaporation of solvent under vacuum to dryness to obtain a crude 2-(2-Furyl)benzimidazole-6-carboxamidoxime (6-AFBD). Crystallization of crude amidoxime derivative using methanol to obtain a crystalline 2-(2-Furyl)benzimidazole-6-carboxamidoxime (6-AFBD). The yield of 6-CFBD is approximately 70%. The proton nuclear magnetic resonance (¹H-NMR) results including chemical shift, δ (given in parts per million) that indicates the type of protons present in the active compound, 6-AFBD is given as follows: δ is 5.8 indicating the presence of amidoxime amine protons, 2H, and δ is 6.7-8.0 indicating the presence of aromatic protons, 6H, 8 is 9.5 indicating the presence of amidoxime hydroxyl proton, 1H and 8 is 13.0 indicating the presence of imidazole-NH, 1H. The solvent used is dimethyl sulfoxide (DMSO-d6).

### Example 6

Biological studies of benzimidazole derivatives with fungi—Aspergillus flavus carried out with respect to the growth and aflatoxin production. For the purpose 2-(2-Furyl) benzimidazole (FBD) stock in DMSO 50 mg/mL added to culture medium to have 50 μg/mL final concentration of compound in the medium (YES). A. flavus spores 5000/mL inoculated into 10 mL medium and incubated at 27° C.±2 for 7 days. At the end of incubation the mycelium met and medium separated by filtration through Whatman filter paper 1. The growth measured in terms of dry mycelium weight by drying the mycelium in dry heat oven at 60° C. Spent medium was extracted with chloroform and after phase separation the chloroform layer collected and concentrated for aflatoxin analysis. Aflatoxin B1 analyses carried out qualitatively and quantitatively by thin layer chromatography and UV spectrophotometry.

### Example 7

Biological studies as in example 6 performed for the other benzimidazole derivatives, 2-(2-Furyl)-6-nitrobenzimidazole (6-NFBD) by substituting FBD with 6-NFBD.

### Example 8

Biological studies as in example 6 performed for the other benzimidazole derivatives, 2-(2-Furyl)benzimidazole-6-carbonitrile (6-CFBD) by substituting FBD with 6-CFBD.

### Example 9

Biological studies as in example 6 performed for the other benzimidazole derivatives, 2-(2-Furyl)benzimidazole-6-carboxylic acid (6-CAFBD) by substituting FBD with 6-CAFBD.

### Example 10

Biological studies as in example 6 performed for the other benzimidazole derivatives, 2-(2-Furyl)benzimidazole-6-carboxamidoxime (6-AFBD) by substituting FBD with 6-AFBD.

### Example 11

Formulation of each benzimidazole derivative from Example 1 to 5-particulate composites (from 50 nanometers to 50 millimeter diameter) of uniform can formulated with additives of various salts into at least one of the formulations, but not limited to, liquids, solutions, concentrates, gels, paste, aerosol sprays, powders, tablets, crystals, pellets, granules, emulsions, seed forms or any other suitable forms.

### DETAILED DESCRIPTION OF THE DRAWINGS

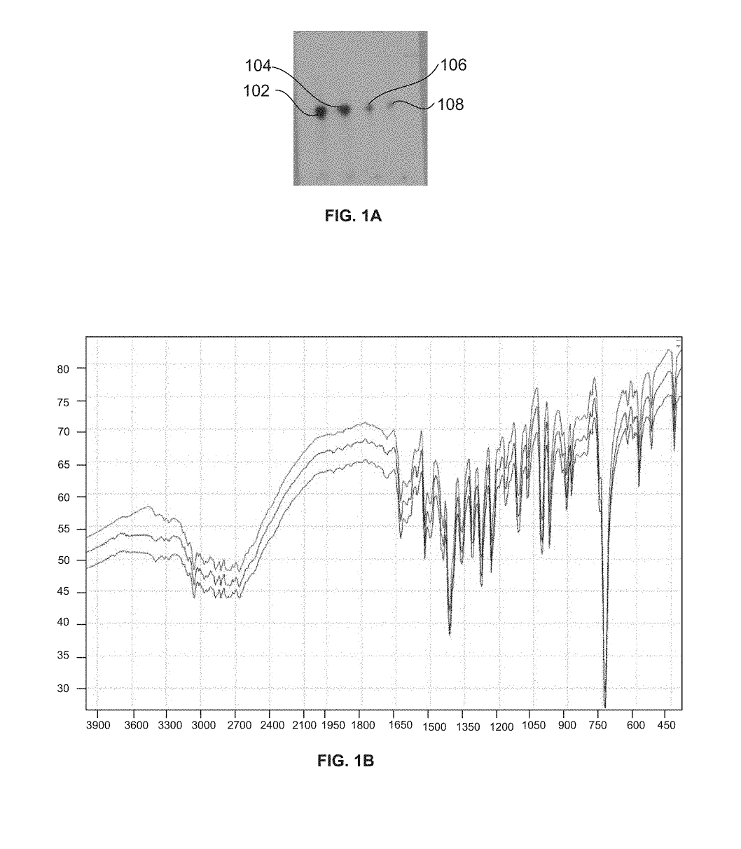

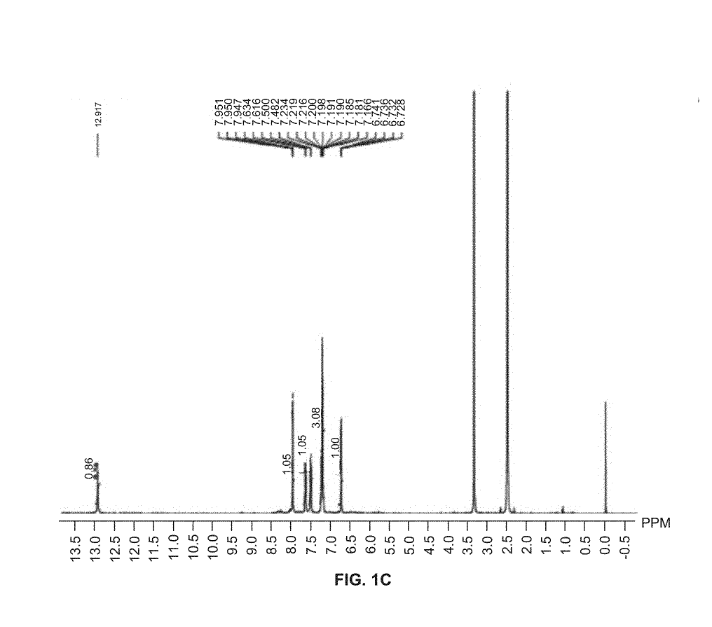

FIGS. 1A-1C are schematic and graphical representations of thin-layer chromatogram, Dose-response overlay IR spectrum and ¹H-NMR spectrum of a benzimidazole derivative, 2-(2-Furyl)benzimidazole (FBD) respectively, in accordance with an embodiment of the present disclosure. In FIG. 1A, the schematic representation of thin-layer chromatogram illustrates spots 102, 104, 106 and 108 representing various concentrations of FBD samples such as 10 mg/ml, 5 mg/ml, 2.5 mg/ml, 1.25 mg/ml that is prepared using chloroform respectively. The spots of the thin-layer chromatogram are detected using iodine chamber and mobile phase used is the mixture of Ethyl acetate and Hexane in 7:3 ratio. In FIG. 1B, the dose-response overlay IR Spectrum of FBD is shown, that is determined by Potassium bromide (KBr) pellet method. In FIG. 1C, the ¹H-NMR Spectrum of the FBD is shown. The analysis carried out and the values obtained by specific experiments are as have been described in previous sections.

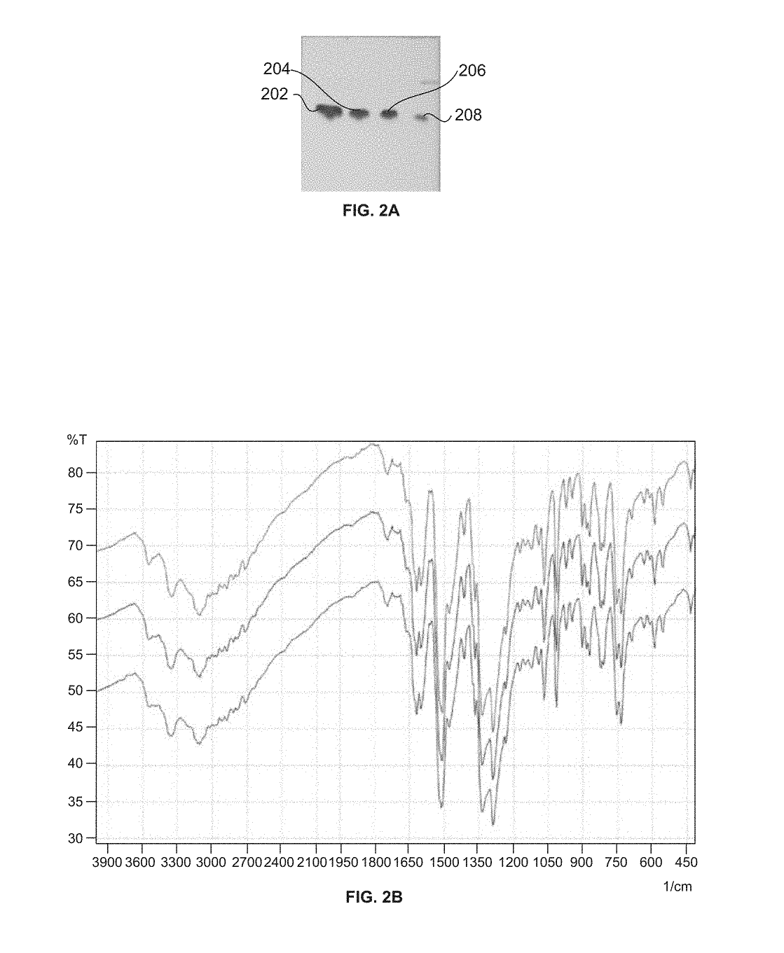

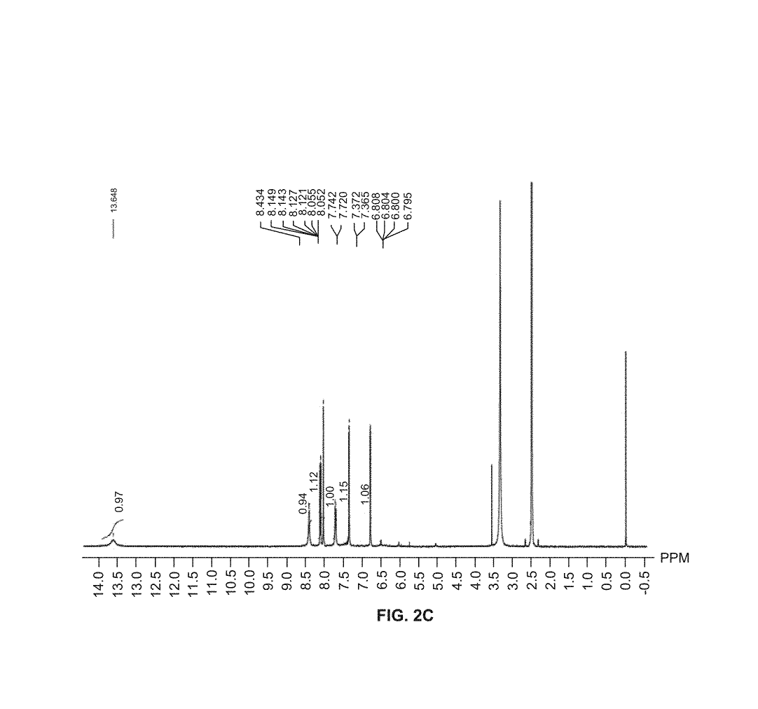

FIGS. 2A-2C are schematic and graphical representation of thin-layer chromatogram, Dose-response overlay IR spectrum and H-NMR spectrum of 2-(2-Furyl)-6-nitrobenzimidazole (6-NFBD) respectively, in accordance with an embodiment of the present disclosure. In FIG. 2A, the schematic representation of the thin-layer chromatogram illustrates spots 202, 204, 206 and 208 representing various concentrations of 6-NFBD samples such as 10 mg/ml, 5 mg/ml, 2.5 mg/ml, 1.25 mg/ml that is prepared using chloroform respectively. The spots of the thin-layer chromatogram are detected using iodine chamber and mobile phase used is the mixture of Ethyl acetate and Hexane in 7:3 ratio. In FIG. 2B, the dose-response overlay IR Spectrum of 6-NFBD is shown, that is determined by Potassium bromide (KBr) pellet method. In FIG. 2C, the ¹H-NMR Spectrum of the 6-NFBD is shown.

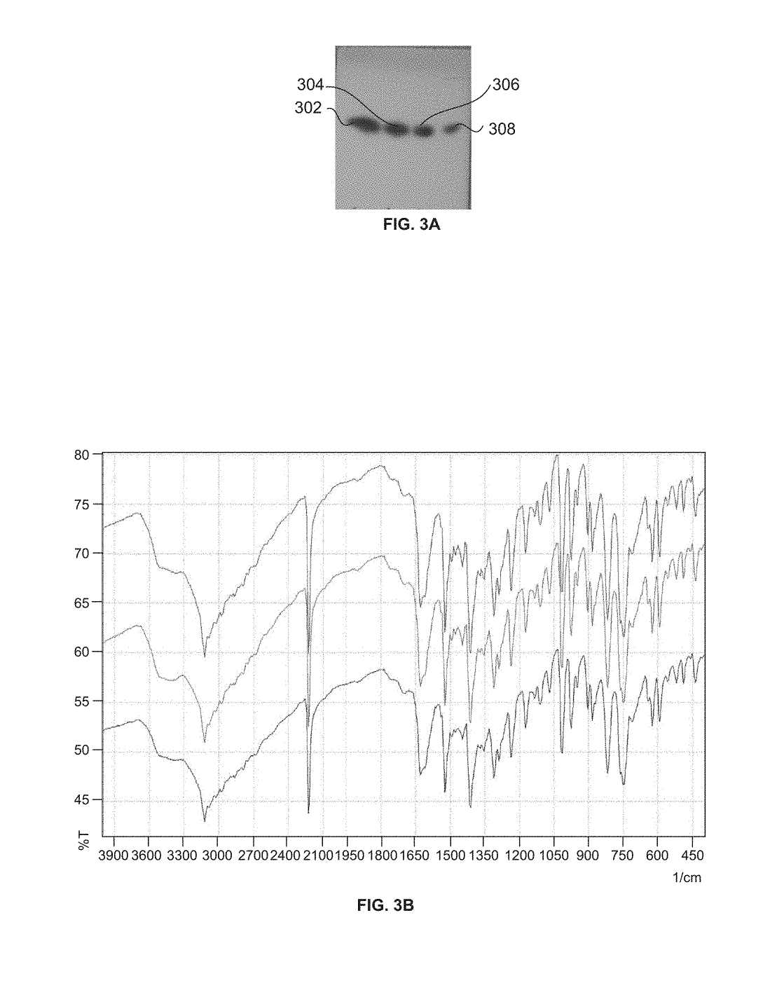

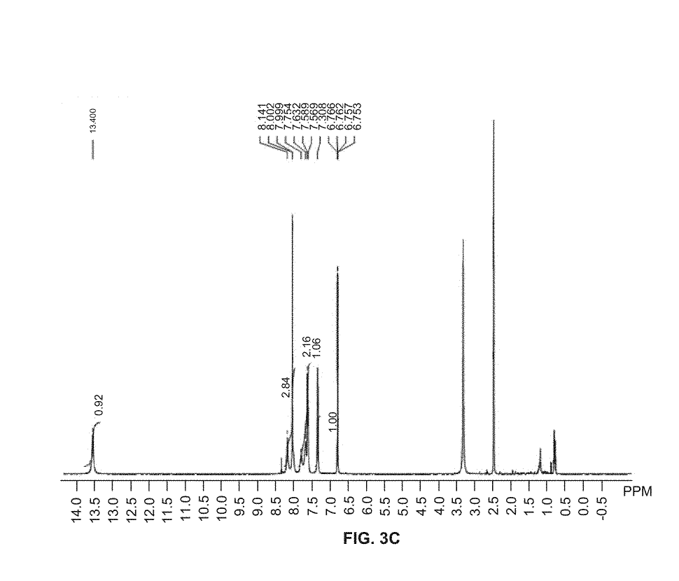

FIGS. 3A-3C are schematic and graphical representation of thin-layer chromatogram, Dose-response overlay IR spectrum and ¹H-NMR spectrum of 2-(2-Furyl)benzimidazole-6-carbonitrile (6-CFBD) respectively, in accordance with an embodiment of the present disclosure. In FIG. 3A, the schematic representation of the thin-layer chromatogram illustrates spots 302, 304, 306 and 308 representing various concentrations of 6-CFBD samples such as 10 mg/ml, 5 mg/ml, 2.5 mg/ml, 1.25 mg/ml that is prepared using chloroform respectively. The spots of the thin-layer chromatogram are detected using iodine chamber and mobile phase used is the mixture of Ethyl acetate and Hexane in 1:1 ratio. In FIG. 3B, the dose-response overlay IR Spectrum of 6-CFBD is shown, that is determined by Potassium bromide (KBr) pellet method. In FIG. 3C, the ¹H-NMR Spectrum of the 6-CFBD is shown.

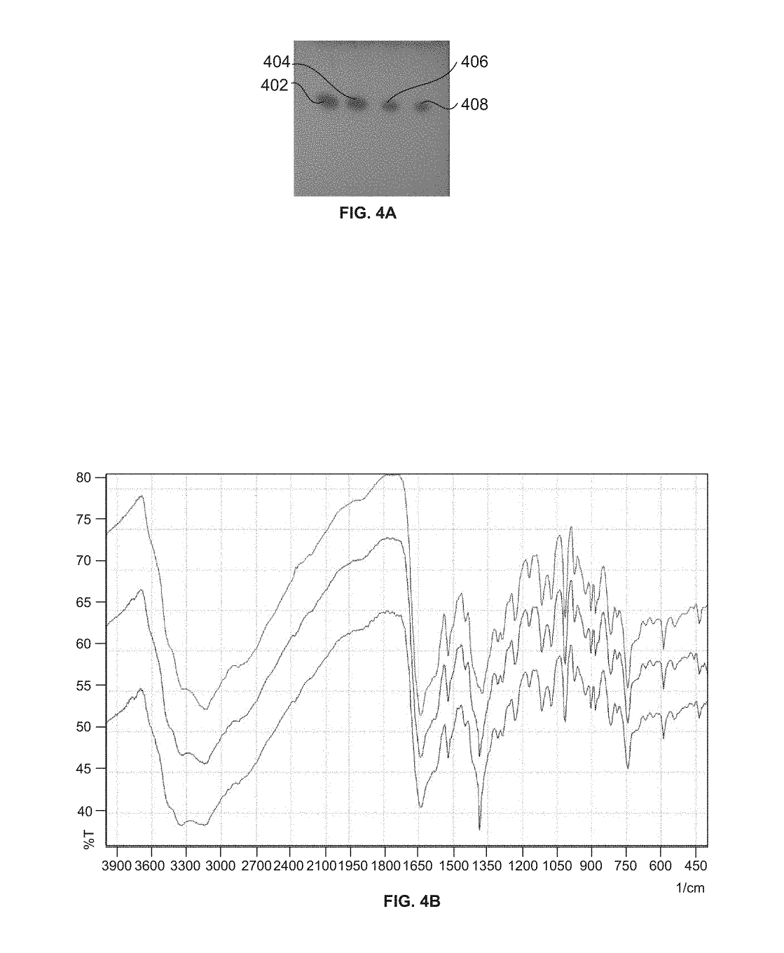

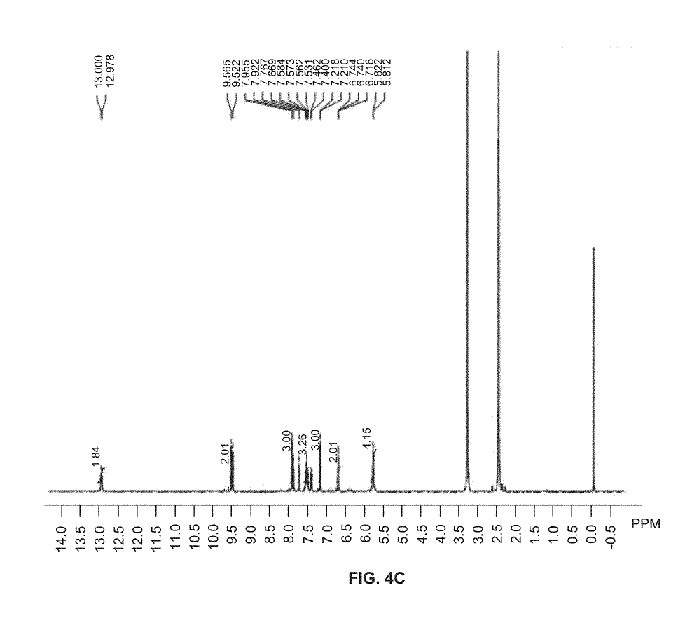

FIGS. 4A-4C are schematic and graphical representation of thin-layer chromatogram, Dose-response overlay IR spectrum and H-NMR spectrum of 2-(2-Furyl)benzimidazole-6-carboxamidoxime (6-AFBD) respectively, in accordance with an embodiment of the present disclosure. In FIG. 4A, the schematic representation of the thin-layer chromatogram illustrates spots 402, 404, 406 and 408 representing various concentrations of 6-AFBD samples such as 10 mg/ml, 5 mg/ml, 2.5 mg/ml, 1.25 mg/ml that is prepared using methanol respectively. The spots of the thin-layer chromatogram are detected using iodine chamber and mobile phase used is the mixture of Ethyl acetate and Hexane in 4:1 ratio. In FIG. 4B, the dose-response overlay IR Spectrum of 6-AFBD is shown, that is determined by Potassium bromide (KBr) pellet method. In FIG. 4C, the ¹H-NMR Spectrum of the 6-AFBD is shown.

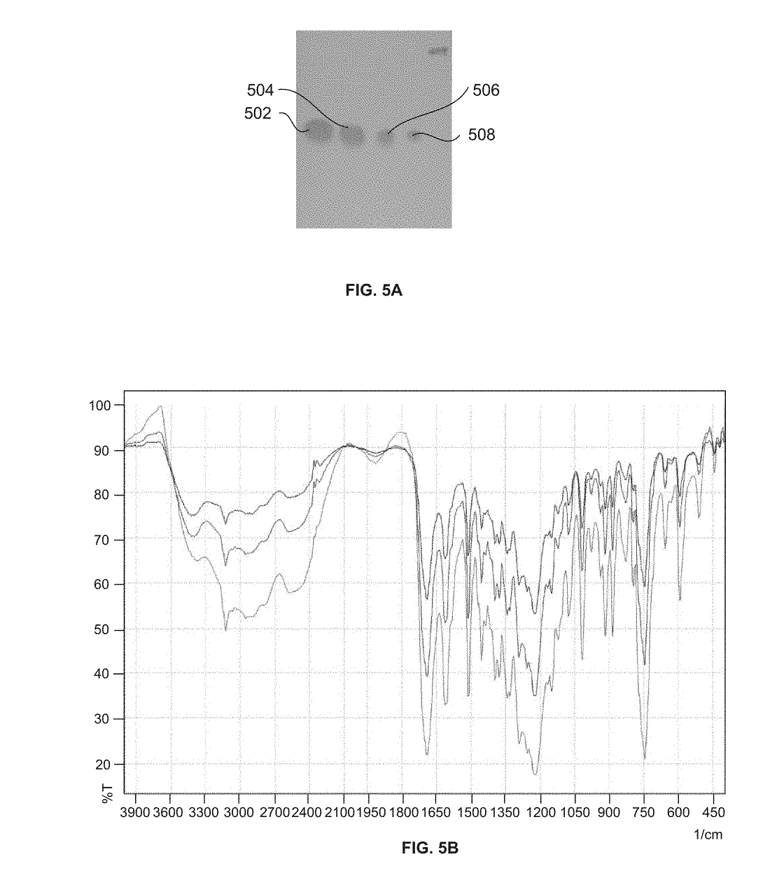

FIGS. 5A-5B are schematic and graphical representation of thin-layer chromatogram, Dose-response overlay IR spectrum and H-NMR spectrum of 2-(2-Furyl)benzimidazole-6-carboxylic acid (6-CAFBD) respectively, in accordance with an embodiment of the present disclosure. In FIG. 5A, the schematic representation of the thin-layer chromatogram illustrates spots 502, 504, 506 and 508 representing various concentrations of 6-CAFBD samples such as 10 mg/ml, 5 mg/ml, 2.5 mg/ml, 1.25 mg/ml that is prepared using methanol respectively. The spots of the thin-layer chromatogram are detected using iodine chamber and mobile phase used is the mixture of Ethyl acetate and Hexane in 8:2 ratio. In FIG. 5B, the dose-response overlay IR Spectrum of 6-CAFBD is shown, that is determined by Potassium bromide (KBr) pellet method.

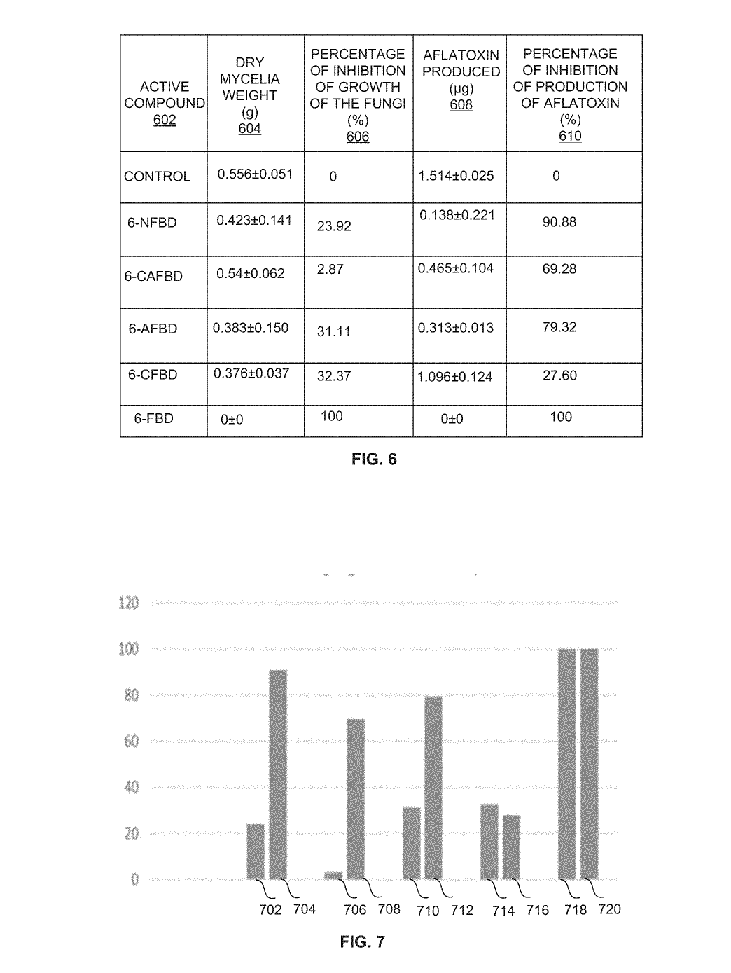

FIG. 6 is a tabular view illustrating an inhibition activity on growth of Aspergillus flavus in yeast extract sucrose (YES) medium in accordance with an embodiment of the present disclosure. The tabular view includes an active compound field 602, a dry mycelia weight field 604, a percentage of inhibition of growth of the fungi field 606, an aflatoxin produced field 608, a percentage of inhibition of production of aflatoxin field 610. The active compound field 602 includes various active compounds and a control that is added to Aspergillus flavus that is grown in yeast extract sucrose (YES) medium for 7 days of incubation. The amount of various active compounds that is added Aspergillus flavus is 50 μg/ml. The various active compounds includes 2-(2-Furyl)-6-nitrobenzimidazole (6-NFBD), 2-(2-Furyl)benzimidazole-6-carboxylic acid (6-CAFBD), 2-(2-Furyl)benzimidazole-6-carboxamidoxime (6-AFBD), 2-(2-Furyl)benzimidazole-6-carbonitrile (6-CFBD) and 2-(2-Furyl)benzimidazole (FBD).

The dry mycelia weight field 604 includes the dry mycelia weight in grams (g) measured after the application of each active compound to the Aspergillus flavus grown in the YES medium. The percentage of inhibition of growth of the fungi field 606 includes the various percentage of inhibition of growth of the fungi, Aspergillus flavus measured after the application of various active compounds. The aflatoxin produced field 608 includes the production of aflatoxin, AFB1 by Aspergillus flavus measured in micrograms (μg). The percentage of inhibition of production of aflatoxin field 610 includes the various percentage of inhibition of production of aflatoxin measured after the application of various active compounds.

From the above tabular view, it is observed that the presence of the 6-NFBD, the 6-CAFBD and the 6-AFBD in the YES medium does not inhibit the growth of the fungi, Aspergillus flavus but the production of aflatoxin, AFB1 was suppressed in comparison to the control i.e. without test compound. The 6-NFBD at the concentration of 50 μg/mL of the YES medium showed about 90% inhibition of production of the AFB1. The active compounds 6-AFBD and the 6-CAFBD show 80% and 70% inhibition of production of AFB1 respectively. However, the active compound 6-CFBD could neither showed inhibition of fungi growth nor the AFB1 production as the growth of the Aspergillus flavus and the production of aflatoxin is almost similar to that of the control. For the active compound, 6-CFBD only 27% inhibition of production of the AFB1 is observed in comparison to control, which is proportionate to the percentage of inhibition of the growth of the Aspergillus flavus (32%). The active compound, FBD showed complete inhibition of both the growth of the fungi and the production of the AFB1, that is 100%.

With reference to the FIG. 6, FIG. 7 is a graphical representation of a percentage of inhibition of Aspergillus flavus growth and aflatoxin production in yeast extract sucrose (YES) medium in accordance with an embodiment of the present disclosure. The percentage of inhibition of growth of the fungi field is marked in X-axis and the percentage of inhibition of production of aflatoxin of each active compound is marked in Y-axis. The percentage of inhibition of growth of the Aspergillus flavus and the percentage of inhibition of production of aflatoxin by 2-(2-Furyl)-6-nitrobenzimidazole is represented as 702 and 704. The percentage of inhibition of growth of the Aspergillus flavus and the percentage of inhibition of production of aflatoxin by the 2-(2-Furyl)benzimidazole-6-carboxylic acid is represented as 706 and 708. The percentage of inhibition of growth of the Aspergillus flavus and the percentage of inhibition of production of aflatoxin by the 2-(2-Furyl)benzimidazole-6-carboxamidoxime is represented as 710 and 712. The percentage of inhibition of growth of the Aspergillus flavus and the percentage of inhibition of production of aflatoxin by the 2-(2-Furyl)benzimidazole-6-carbonitrile is represented as 714 and 716. The percentage of inhibition of growth of the Aspergillus flavus and the percentage of inhibition of production of aflatoxin by the 2-(2-Furyl)benzimidazole is represented as 718 and 720.

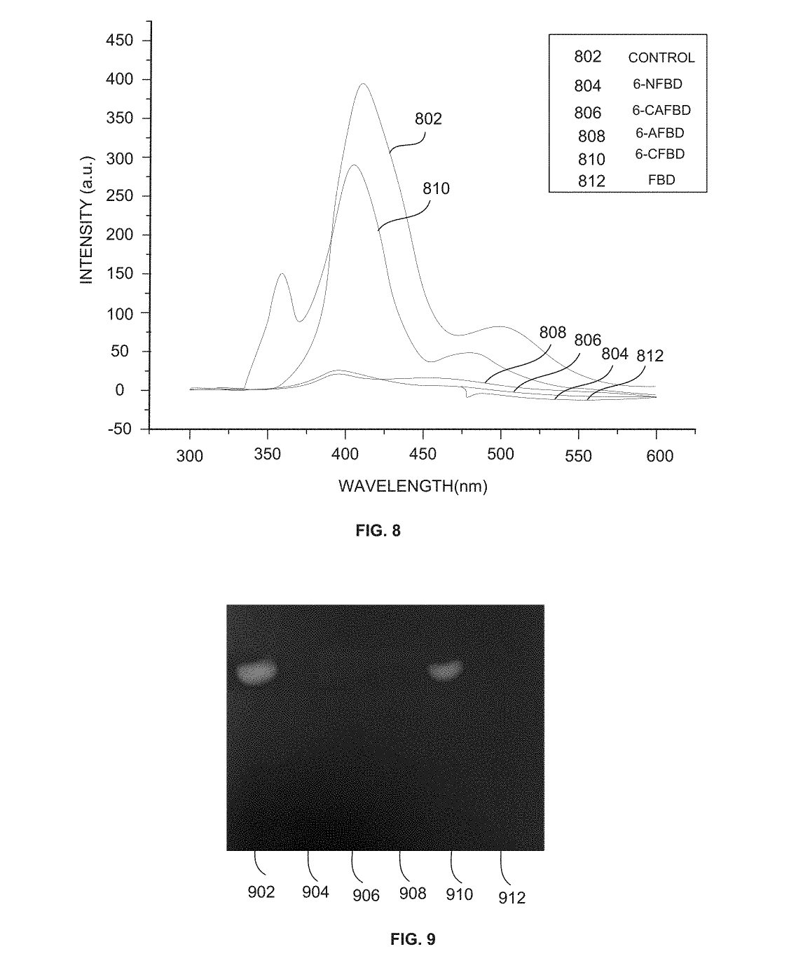

FIG. 8 is a graphical representation of Fluorescence overlay chromatogram showing inhibition of an aflatoxin B1 (AFB1) in YES medium in accordance with an embodiment of the present disclosure. The graphical representation includes wavelength (nm) in X-axis and intensity (AU) in Y-axis. The fluorescence intensity of the YES medium is measured using a fluorescence spectroscopy for analysing production of the AFB1. The graph shows the peaks 802, 804, 806, 808, 810 and 812 for the control, 6-NFBD, 6-CAFBD, 6-AFBD, 6-CFBD and FBD respectively. The spectroscopic analysis showed a decrease in fluorescence at 430 nm which is the characteristic for the AFB1 in presence of various active compounds.

FIG. 9 is a representation of thin layer chromatogram showing an aflatoxin B1 (AFB1) AFB1 spots in accordance with an embodiment of the present disclosure. The schematic representation shows the wells for 902, 904, 906, 908, 910 and 912 for the control, 6-NFBD, 6-CAFBD, 6-AFBD, 6-CFBD and FBD respectively. The AFB1 spots are clearly displayed in bands for the control well 902 and for the 6-CFBD 910.

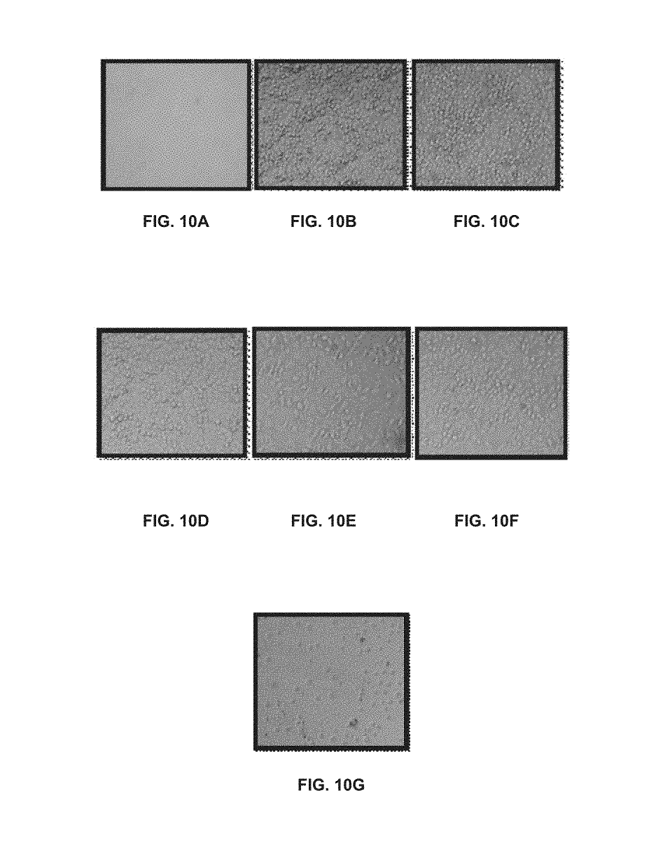

FIGS. 10A-10G is a microscopic view of a HeLa cell line cultured with test compound in accordance with an embodiment of the present disclosure. The schematic representation 10A-10G shows the microscopic view of a HeLa cell line culture of animal cells in the presence of no active compounds (a blank), a control, 2-(2-Furyl)benzimidazole (FBD), 2-(2-Furyl)benzimidazole-6-carboxylic acid (6-CAFBD), 2-(2-Furyl)benzimidazole-6-carboxamidoxime (6-AFBD), 2-(2-Furyl)-6-nitrobenzimidazole (6-NFBD), and a positive control respectively. The positive control is prepared by adding aflatoxin (AFB1) in Dulbecco's modified Eagle's medium (DMEM, HiMedia, India) and 10% Fetal bovine serum (FBS).

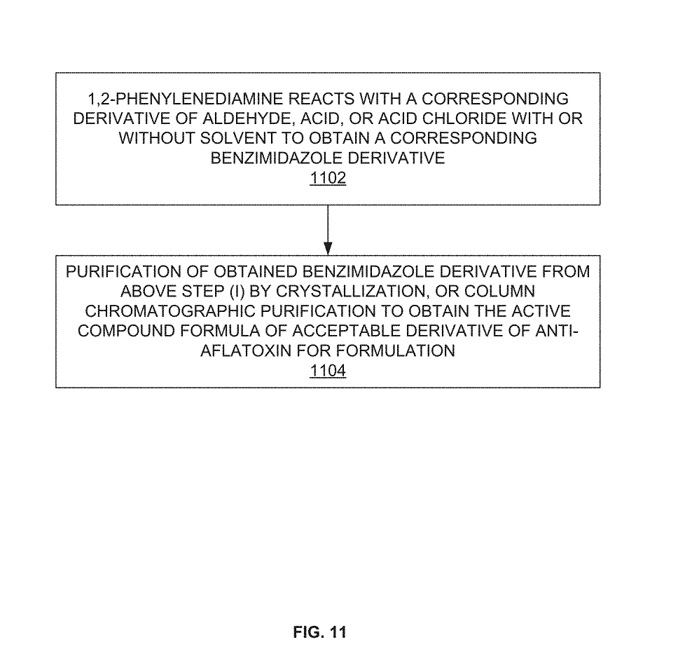

FIG. 11 is a flowchart illustrating a method of preparation of an active compound in accordance with an embodiment of the present disclosure. In step 1102, 1,2-phenylenediamine reacts with a corresponding derivative of aldehyde, acid, or acid chloride with or without solvent to obtain a corresponding benzimidazole derivative. In step 1104, purification of the obtained benzimidazole derivative from the above step 1102 is performed by crystallization, or column chromatography to obtain an active compound formula of acceptable derivative of anti-aflatoxin for formulation.

Based upon the foregoing disclosure, it should now be apparent that the method of preparing the active compounds as described herein will carry out the objects set forth hereinabove. It is, therefore, to be understood that any variations evident fall within the scope of the claimed invention and thus, the selection of specific component elements can be determined without departing from the spirit of the invention herein disclosed and described.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A composition for inhibiting synthesis of mycotoxins without affecting said growth of fungi comprising a benzimidazole compound of Formula, 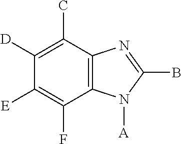
where A is hydrogen or C1-C6 alkyl or C6-C14 aryl; B is C1-C10 alkyl or C6-C14 aryl or oxygen, sulfur, nitrogen containing heteroaryl; C and D are hydrogen or C1-C6 alkyl or C1-C6 alkoxyl or C6-C14 aryl; E is hydrogen or nitro or cyano or carboxyl or acetamidoxime or amidoxime or C1-C6 alkyl or C1-C6 alkoxyl or C6-C14 aryl or oxygen, sulfur, and nitrogen containing heteroaryl; F is hydrogen or C1-C6 alkyl or C1-C6 alkoxyl or C6-C14 aryl or oxygen, sulfur, and nitrogen containing heteroaryl, and mixture thereof with corresponding salts.

2. The composition of claim 1, wherein when B is 2-furyl and A, C, D, E, F are hydrogen (—H), said compound of Formula is 2-(2-Furyl)benzimidazole (FBD) as follows:

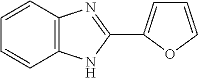 [SMILES: c1coc(-c2nc3ccccc3[nH]2)c1]

3. The composition of claim 1, wherein when B is 2-furyl and E is nitro (—NO2) and A, C, D, F are hydrogen (—H), said compound of Formula is 2-(2-Furyl)-6-nitrobenzimidazole (6-NFBD) as follows:

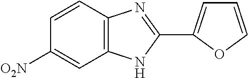 [SMILES: O=[N+]([O-])c1ccc2nc(-c3ccco3)[nH]c2c1]

4. The composition of claim 1, wherein when B is 2-furyl and E is carbonitrile (—CN) and A, C, D, F are hydrogen (—H), said compound of Formula is 2-(2-Furyl) benzimidazole-6-carbonitrile (6-CFBD) as follows:

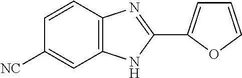 [SMILES: N#Cc1ccc2nc(-c3ccco3)[nH]c2c1]

5. The composition of claim 1, wherein when B is 2-furyl and E is carboxyl (—COOH) and A, C, D, F are hydrogen (—H), said compound of Formula is 2-(2-Furyl) benzimidazole-6-carboxylic acid (6-CAFBD):

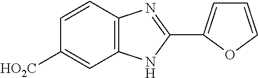 [SMILES: O=C(O)c1ccc2nc(-c3ccco3)[nH]c2c1]

6. The composition of claim 1, wherein when B is 2-furyl and E is acetamidoxime (—C(NH2)=NOH) and A, C, D, F are hydrogen (—H), said compound of Formula is 2-(2-Furyl) benzimidazole-6-carboxamidoxime (6-AFBD) as follows:

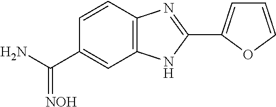

7. The composition of claim 1, wherein said composition is impregnated as sachet, packaging material, patch, or air-freshener.

8. The composition of claim 1, wherein said composition is encapsulated with liposomes and related matrices.

9. An anti-aflatoxin composition comprising one or more active compounds selected from a group comprising:
(a) 2-(2-Furyl)-6-nitrobenzimidazole,
(b) 2-(2-Furyl)benzimidazole,
(c) 2-(2-Furyl)benzimidazole-6-carbonitrile,
(d) 2-(2-Furyl)benzimidazole-6-carboxamidoxime,
(e) 2-(2-Furyl)benzimidazole-6-carboxylic acid, or
(f) combinations thereof.

10. The anti-aflatoxin composition as claimed in claim 9, wherein the one or more active compounds ranges from 0.001%-60%.

11. The anti-aflatoxin composition of claim 9, wherein said anti-aflatoxin composition further comprises at least one of diluents, additives or any other carrier which is compatible with the at least one of the active compound.

12. The anti-aflatoxin composition of claim 9, wherein said anti-aflatoxin composition is formulated as liquids, gels, sprays, powders, crystals, pellets, granules, emulsions or seed forms or its derivative thereof.

13. The anti-aflatoxin composition of claim 9, wherein for prophylaxis or treatment of agricultural products, food grains, dairy products, fermented products, or subject treatment of mycotoxins, said active compound of claim 9 is an effective amount.

14. The anti-aflatoxin composition of claim 9, wherein said anti-aflatoxin inhibits the mycotoxin synthesis during fermentation process or semisynthetic process that is used for brewing beverages, pharmaceuticals, biopharmaceuticals, and biosimilars.

15. The anti-aflatoxin composition of claim 14, wherein said anti-aflatoxin is formulated as liquids, gels, sprays, powders, crystals, pellets, granules, emulsions or seed forms or its derivatives thereof.

16. The anti-aflatoxin composition of claim 14, wherein said anti-aflatoxin composition is impregnated as sachet, packaging material, patch, or air-freshener.

17. A method for inhibiting said synthesis of mycotoxin without affecting said growth of said fungi by applying a composition comprising an active compound comprising a benzimidazole, wherein said process for said preparation of benzimidazoles comprising said steps of
i) Corresponding derivatives of 1,2-phenylenediamine reacts with corresponding derivative of aldehyde, acid, or acid chloride with or without solvent (nitrobenzene) to obtain said corresponding benzimidazole derivative of formula. 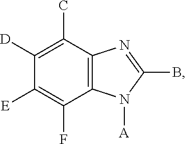
and
ii) Purification of obtained benzimidazole derivative from above step (i) by crystallization, or column chromatographic purification to obtain the active compound Formula of acceptable derivative of anti-aflatoxin for formulation.

18. The method of claim 17, wherein said anti-aflatoxin is formulated as liquids, gels, sprays, powders, crystals, pellets, granules, emulsions or seed forms or its derivatives thereof.

19. The method of claim 17, wherein said anti-aflatoxin encapsulated with liposomes and related matrices.

20. The method of claim 17, wherein said anti-aflatoxin immobilized or covalently coupled with polymeric resins and carriers thereof.
